(19) United States
(12) Patent Application Publication
(10) Pub. No.: US 2010/0267734 A1
(43) Pub. Date: Oct. 21, 2010

(54) N-[(3,5-DICHLOROPHENYL)METHYL]-2-(4-METHYL-1-PIPERAZINYL)-2-(1-NAPHTHALENYL)ETHANAMIDE AS GHRELIN RECEPTOR MODULATOR

(75) Inventor: Thomas Daniel Heightman, Harlow (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited, Greenford (GB)

(21) Appl. No.: 12/602,834

(22) PCT Filed: Jun. 5, 2008
(86) PCT No.: PCT/EP2008/057021

§ 371 Date: Jun. 23, 2010

(30) Foreign Application Priority Data
Jun. 6, 2007 (GB) 0710863.2

(51) Int. Cl. A61K 31/495; C07D 241/04; A61P 3/00

(52) U.S. Cl. 514/252.12; 544/400

(57) ABSTRACT

The present invention relates to novel compounds of formula (I) or a pharmaceutically acceptable salt or solvate thereof,

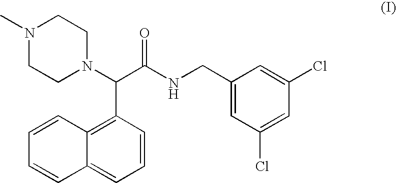

processes for their preparation, intermediates useable in these processes, pharmaceutical compositions containing them and their use in therapy, for example as modulators of the growth hormone secretagogue receptor (also referred to as the ghrelin receptor or GHSR1a receptor) and/or for the treatment and/or prophylaxis of a disorder mediated by the ghrelin receptor.

The present invention relates to novel amide compounds, processes for their preparation, intermediates usable in these processes, and pharmaceutical compositions containing the compounds. The invention also relates to the use of the amide compounds in therapy, for example as modulators of the growth hormone secretagogue receptor (also referred to as the ghrelin receptor or GHSR1a receptor) and/or for the treatment and/or prophylaxis of eating disorders such as a binge eating disorder.

Ghrelin is the endogenous ligand for the growth hormone (GH) secretagogue receptor. It was originally purified from stomach and is a 28 amino acid peptide hormone in which the serine at position 3 is n-octanoylated. It has potent GH releasing activity and thus is believed to play an important role in maintaining GH release and energy homeostasis. In particular, it appears to exert potent appetite-stimulating activities.

It is therefore desirable to find new compounds which modulate ghrelin receptor activity.

According to the invention there is provided a compound of formula (I) or a pharmaceutically acceptable salt or solvate thereof:

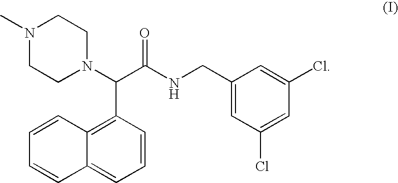

In one embodiment there is provided a compound of formula (I)

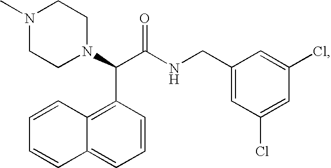

or a pharmaceutically acceptable salt or solvate thereof.

Salts of the compounds of the present invention are also encompassed within the scope of the invention. Because of their potential use in medicine, the salts of the compounds of formula (I) are preferably pharmaceutically acceptable. Suitable pharmaceutically acceptable salts can include acid addition salts. A pharmaceutically acceptable acid addition salt can be formed by reaction of a compound of formula (I) with a suitable inorganic or organic acid (such as hydrobromic, hydrochloric, hydroiodic, sulfuric, nitric, phosphoric, p-toluenesulfonic, methanesulfonic or naphthalenesulfonic acid), optionally in a suitable solvent such as an organic solvent, to give the salt which is usually isolated for example by crystallisation and filtration. Examples of pharmaceutically acceptable acid addition salts of a compound of formula (I) include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497. Other salts, which are not pharmaceutically acceptable, for example the trifluoroacetate salt, may be useful in the preparation of compounds of this invention and these form a further aspect of the invention. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Compounds hereafter using appropriate isotopic variations of suitable reagents.

In a further embodiment the present invention thus also provides compounds of formula (I) and pharmaceutically acceptable salts or solvates thereof, for use in medical therapy, and particularly in the treatment of disorders mediated by the ghrelin receptor.

In a further embodiment the present invention is directed to methods of modulating ghrelin receptor activity for the prevention and/or treatment of disorders mediated by the ghrelin receptor.

In a further embodiment the present invention provides a method of treatment of a mammal suffering from a disorder mediated by the ghrelin receptor, which comprises administering to said subject an effective amount of a compound of formula (I) or a pharmaceutically acceptable salt or solvate thereof. Such treatment comprises the step of administering a therapeutically effective amount of the compound of formula (I), including a pharmaceutically acceptable salt or solvate thereof. Such treatment can also comprise the step of administering a therapeutically effective amount of a pharmaceutical composition containing a compound of formula (I), including a pharmaceutically acceptable salt or solvate thereof. As used herein, the term “treatment” refers to alleviating the specified condition, eliminating or reducing the symptoms of the condition, slowing or eliminating the progression of the condition, and preventing or delaying the reoccurrence of the condition in a previously afflicted patient or subject.

A further embodiment of the present invention provides the use of a compound of formula (I), or a pharmaceutically acceptable salt or solvate thereof, in the preparation of a medicament for the treatment of a disorder mediated by the ghrelin receptor.

A further embodiment of the present invention provides the use of a compound of formula (I), or a pharmaceutically acceptable salt or solvate thereof, for the treatment of a disorder mediated by the ghrelin receptor.

The action of the endogenous ligand ghrelin at the ghrelin receptor has been shown to result in potent growth-hormone releasing activity, appetite stimulation, stimulation of gastric motility and acid secretion, positive cardiovascular effects and direct action on bone formation. Thus, a ghrelin receptor modulator may achieve a beneficial effect in the treatment of growth-hormone deficiencies, eating disorders, gastrointestinal disease, cardiovascular diseases, osteoporosis, aging and catabolic states or chronic wasting syndromes (Kojima and Kangawa, Nature Clincal Practice, February 2006, Vol 2, No. 2, 80-88). A ghrelin receptor modulator may also achieve a beneficial effect in the treatment of sleep disorders (Brain Research, 1088 (2006) 131-140).

Particular disorders which are associated with the ghrelin receptor and thus may be mediated by the ghrelin receptor such that a ghrelin receptor modulator may achieve a beneficial effect include obesity and risk factors associated with obesity, including but not limited to diabetes, complications associated with diabetes, metabolic syndrome, cardiovascular disorders (including atherosclerosis and dyslipidemia).

Other diseases and/or conditions mediated by the ghrelin receptor wherein a ghrelin include the following, treating a growth hormone deficient state, increasing muscle mass, increasing bone density, treating sexual disfunction in males and females, facilitating a weight gain, facilitating weight maintenance, facilitating appetite increase (for example facilitating weight gain, maintenance or appetite increase is useful in a patient having a disorder, or under going a treatment, accompanied by weight loss). Examples of diseases or disorders accompanied by weight loss include anorexia, bulimia, cancer cachexia, AIDS, wasting, cachexia and wasting in frail elderly. Examples of treatments accompanied by weight loss include chemiotherapy, radiation therapy, temporary or permanent immobilisation, and dialysis.

Further diseases or conditions include sleep disorders, congestive heart failure, metabolic disorder, improvements in memory function, breast cancer, thyroid cancer, ameliorating ischemic nerve or muscle damage.

The compounds of the invention function by modulating the activity of the ghrelin receptor. They may activate/inactivate the receptor by acting as an agonist, partial agonist, inverse agonist, antagonist or partial antagonist.

Eating disorders include Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50). [the numbers in brackets after the listed diseases above refer to the classification code in Diagnostic and Statistical Manual of Mental Disorders, 4th Edition, published by the American Psychiatric Association (DSM-IV) and/or the International Classification of Diseases, 10th Edition (ICD-10)].

In a further embodiment the invention provides the use of a compound of formula (I), or a pharmaceutically acceptable salt or solvate thereof, in the preparation of a medicament for the treatment of an eating disorder

In a further embodiment the present invention provides a method of treatment of a mammal suffering from an eating disorder which comprises administering to said subject an effective amount of a compound of formula (I) or a pharmaceutically acceptable salt or solvate thereof.

Gastrointestinal diseases include gastric ileus, gastric ulcer and inflammatory bowel diseases such as Crohn's disease and ulcerative colitis. The compounds of the invention may also be useful for treatments to alleviate symptoms associated with gastro-esophageal reflus and/or with dyspepsia, with or without appetite-/metabolic-related cachexia, and in the treatment of paralytic ileus or pseudo obstruction, and of conditions associated with constipation, such as constipation-predominant irritable bowel syndrome.

Cardiovascular diseases include heart failure and dilated cardiomyopathy.

Catabolic states or chronic wasting syndromes may be seen in post-operative patients and also include AIDS-associated and cancer-associated wasting syndromes, such as cancer cachexia.

While it is possible that, for use in therapy a therapeutically effective amount of a compound of formula (I), or a pharmaceutically acceptable salt or solvate thereof, may be administered as the raw chemical, it is possible to present the active ingredient as a pharmaceutical composition. Thus, in a further embodiment the invention provides a pharmaceutical composition comprising a compound of formula (I), or a pharmaceutically acceptable salt or solvate thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of (I), or a pharmaceutically acceptable salt or solvate thereof, with one or more pharmaceutically acceptable carriers, diluents or excipients.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

A therapeutically effective amount of a compound of formula (I), or a pharmaceutically acceptable salt or solvate thereof will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the ghrelin receptor will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt or solvate thereof, may be determined as a proportion of the effective amount of the compound of formula (I) per se.

A compound of formula (I), or a pharmaceutically acceptable salt or solvate thereof for use in the instant invention may be used in combination with one or more other therapeutic agents. The invention thus provides in a further embodiment a combination comprising a compound of formula (I), or a pharmaceutically acceptable salt or solvate thereof together with a further therapeutic agent, which may be for example an additional anti-obesity agent. In a yet further embodiment the invention also provides the use of a combination comprising a compound of formula (I), or a pharmaceutically acceptable salt or solvate thereof with a further therapeutic agent in the treatment of disorders mediated by the ghrelin receptor.

When a compound of formula (I), or a pharmaceutically acceptable salt or solvate thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

All publications, including but not limited to patents and patent applications, cited in this specification are herein incorporated by reference as if each individual publication were specifically and individually indicated to be incorporated by reference herein as though fully set forth.

### EXPERIMENTAL

The invention is illustrated by the Compounds described below.

Compounds are named using ACD/Name PRO6.02 chemical naming software (Advanced Chemistry Development Inc., Toronto, Ontario, M5H2L3, Canada).

“Enantiomer 1” indicates a single enantiomer of unknown absolute stereochemistry.

Proton Magnetic Resonance (NMR) spectra were recorded either on Varian instruments at 300, 400 or 500 MHz, or on a Bruker instrument at 300 or 400 MHz. Chemical shifts are reported in ppm (δ) using the residual solvent line as internal standard. Splitting patterns are designed as s, singlet; d, doublet; t, triplet; q, quartet; m, multiplet; br, broad. The NMR spectra were recorded at a temperature ranging from 25 to 90° C.

Mass spectra (MS) were taken on an Agilent MSD 1100 Mass Spectrometer, operating in ES (+) and ES (−) ionization mode or on an Agilent LC/MSD 1100 Mass Spectrometer, operating in ES (+) and ES (−) ionization mode coupled with HPLC instrument Agilent 1100 Series [LC/MS-ES (+/−): analysis performed using a Supelcosil ABZ+ Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [MeCN] in 5 min, finally under these conditions for 2 min; T=40° C.; flux=1 ml/min; LC/MS-ES (+/−):analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [MeCN] in 5 min, finally under these conditions for 2 min; T=40° C.; flux=1 ml/min]. In the mass spectra only one peak in the molecular ion cluster is reported. Total ion current (TIC) and DAD UV chromatographic traces together with MS and UV spectra associated with the peaks were taken also on a HPLC/MS Acquity™ system equipped with 2996 PDA detector and coupled to a Waters

Micromass ZQ™ mass spectrometer operating in positive or negative electrospray ionisation mode. [LC/MS-ES (+/−): analyses performed using an Acquity™ HPLC BEH C18 column (50×21 mm, 1.7 μm particle size), column temperature 40° C. (mobile phase: A-water+0.1% HCOOH/B-MeCN+0.075% HCOOH, Flow rate: 1.0 ml/min, Gradient: t=0 min 3% B, t=0.05 min 6% B, t=0.57 min 70% B, t=1.4 min 99% B, t=1.45 min 3% B)].

For the chiral separation and the chiral quality control two different techniques were used: 1) Supercritical Fluid Choromatography (SFC): analytical chromatography was performed on a Berger SFC Analytix, while for the preparative SFC, a Gilson SFC series SF3 was used 2) High Performance Liquid Chromatography (HPLC): chiral Preparative HPLC was performed using a Waters 600 HPLC system and Agilent series 1100 instrument, while for analytical chromatography an Agilent series 1100 HPLC was used.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be—Si pre-packed cartridges or over pre-packed Biotage silica cartridges.

SPE-SCX cartridges are ion exchange solid phase extraction columns by supplied by Varian. The eluent used with SPE-SCX cartridges is methanol followed by ammonia solution in methanol.

Oasis HLB LP extraction cartridges are ion exchange solid phase extraction columns by supplied by Waters. The eluent used with HLB cartridges is water followed by methanol.

In a number of preparations purification was performed using either Biotage manual flash chromatography (Flash+) or automatic flash chromatography (Horizon) systems. All these instruments work with Biotage Silica cartridge.

SPE-SI cartridges are silica solid phase extraction columns supplied by Varian or IST Isolute.

Where a percentage yield has been quoted for a single enantiomer isolated from a racemic mixture this has been calculated on the basis that the maximum possible yield of material (i.e. 100%) is half of the total mass of the racemate.

Comparative vibrational circular dichroism (VCD) was performed as follows:
Spectrometer: Bomem ChiralIR VCD spectrometer operated at 4 cm⁻¹
Frequency Range: 2000-800
PEM Calibration: PEM calibrated at 1400 cm⁻¹
Scan Method: Three 120-minute block scans per enantiomer.
Solvent(s): CDCl₃
Concentration(s): [ ] ˜4 mg/150 ul
Baseline Correction Method: enantiomer correction method
Additional Processing: Savitsky-Golay 9-point smooth with manual baseline correction

### Abbreviations:

AcOEt: ethyl acetate
DCM: dichloromethane
Et₂O: diethyl ether
MeCN: acetonitrile
TBTU: O-(Benzotriazol-1-yl)-N,N,N′,N′-tetramethyluronium tetrafluoroborate
DMF: dimethylformamide
CCl₄: carbon tetrachloride
DIPEA: diisopropylethylamine

In the procedures that follow, after each starting material, reference to a Description or Example by number is typically provided. This is provided merely for assistance to the skilled chemist. The starting material may not necessarily have been prepared from the batch referred to.

### Intermediate 1: (±)-(4-methyl-1-piperazinyl)(1-naphthalenyl)acetic acid

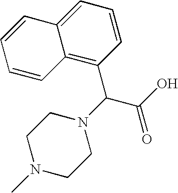

452 μl of N-methylpiperazine (4.07 mmole, Aldrich) were added to a solution of 700 mg of 1-napthaleneboronic acid (4.07 mmole, Alfa Aesar) and 375 mg of glyoxylic acid monohydrate (4.07 mmole, Aldrich) in 7 ml of MeCN. After the addiction of N-methylpiperazine the mixture was refluxed for 24 hours. The solution became dark with formation of a beige precipitate. The reaction was cooled to room temperature and poured into AcOEt (14 ml). The precipitate was filtered and dried in vacuo. Then it was triturated with Et₂O for 1 hour to give the title compound as a beige solid (1.11 g, 96%). ¹H NMR (400 MHz, d⁶DMSO) δ 2.09-2.21 (3H, s), 2.23-2.71 (7H, m), 2.93-3.00 (1H, m), 4.66-4.74 (2H, m), 7.45-7.59 (3H, m), 7.65 (1H, d), 7.90 (2H, dd), 8.54 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B in 1 min., 95% to 0% B in 0.1 min., run time 4.5 min., flow rate: 2 ml/min]. Rₜ=0.261 min. (m/z=285 (M+H)⁺).

### Compound 1: (±)-N-[(3,5-dichlorophenyl)methyl]-2-(4-methyl-1-piperazinyl)-2-(1-naphthalenyl)ethanamide

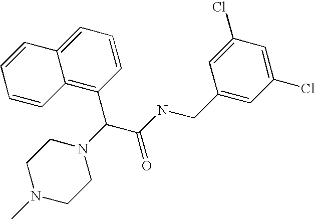

To a suspension of 1.11 g of (4-methyl-1-piperazinyl)(1-naphthalenyl)acetic acid (intermediate 1, 3.9 mmole) in DMF (10 ml), were added 1.36 ml of DIPEA (7.82 mmole, Aldrich) and 1.5 g of TBTU (4.69 mmole, Fluka). After 10 minutes were added 0.625 ml of 3,5-dichlorobenzylamine (4.69 mmole, Maybridge). The solution became clear and dark and it was stirred at room temperature for two days. Then it was diluted with AcOEt and washed with a saturated solution of NaHCO₃ (20 ml). The phases were separated and the organic layer was extracted with AcOEt (3×30 ml). The combined organic layers were washed again with water and ice (2×50 ml), dried over Na₂SO₄ and concentrated to dryness to give the crude compound that was purified by flash chromatography on 25 g silica gel cartridge, using a gradient of DCM/MeOH 10/0 to 8/2 as an eluent. Solvents were removed under reduced pressure to give the title compound as a beige foam (782 mg, 46%). ¹H NMR (400 MHz, CDCl₃) δ 2.25-2.37 (3H, m), 2.38-2.78 (8H, m), 4.36 (1H, dd), 4.49 (1H, dd), 4.76-4.82 (1H, m), 6.96-7.01 (2H, m), 7.20-7.25 (1H, m), 7.29-7.39 (1H, m), 7.43-7.63 (4H, m), 7.86 (2H, dd), 8.37 (1H, d).

### Intermediate 2: N-[(3,5-dichlorophenyl)methyl]-2-(4-methyl-1-piperazinyl)-2-(1-naphthalenyl)ethanamide di-O,O′-p-toluoyl-L-tartaric salt—enantiomer 1

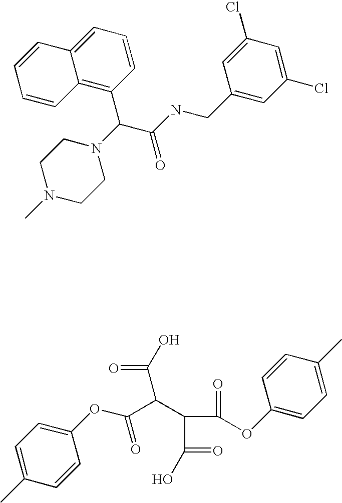

A solution of 685 mg of (−)-di-O,O′-p-toluoyl-L-tartaric acid (1.77 mmole, Fluka) in 4 ml of acetone was added dropwise to a solution of Compound 1 ((±)-N-[(3,5-dichlorophenyl)methyl]-2-(4-methyl-1-piperazinyl)-2-(1-naphthalenyl)ethanamide, 782 mg, 1.77 mmole) in 7 ml of acetone. The reaction was stirred for 3 hours to complete precipitation of the salt. The white precipitate was filtered and dried under vacuum to give 522 mg of a white powder. Then it was suspended in 5 ml of acetone, heated at reflux for 2 hours and then cooled to room temperature overnight. The precipitate was filtered and dried to give the title compound as a white solid (354 mg, 48%). ¹H NMR (400 MHz, d⁶DMSO) δ 2.01-2.14 (3H, m), 2.26-2.40 (6H, m), 2.58-2.74 (2H, m), 2.74-3.05 (2H, m), 3.06-3.62 (4H, m), 4.16-4.38 (2H, m), 4.81-4.85 (1H, m), 5.66-5.66 (2H, m), 7.09 (2H, d), 7.30 (4H, d), 7.38-7.42 (1H, m), 7.46-7.59 (3H, m), 7.68 (1H, d), 7.82 (4H, d), 7.87-7.98 (2H, m), 8.53 (1H, d), 8.77-8.86 (1H, m); Chiral HPLC [Chiralcel OD, 25×4.6 cm. Flow rate: 10.8 ml/min. UV detection: 225 nm. Mobil phase: nHexane/ethanol 70/30]: Rₜ=5.625 min (98.28% ee).

### Compound 2: (2R)-N-[(3,5-dichlorophenyl)methyl]-2-(4-methyl-1-piperazinyl)-2-(1-naphthalenyl)ethanamide

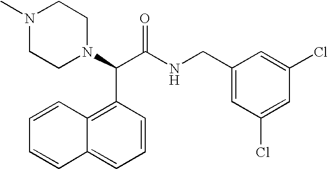

354 mg of intermediate 2 ((N-[(3,5-dichlorophenyl)methyl]-2-(4-methyl-1-piperazinyl)-2-(1-naphthalenyl)ethanamide di-O,O′-p-toluoyl-L-tartaric salt—enantiomer 1, 0.428 mmole) were partitioned between DCM (7 ml) and a saturated solution of K₂CO₃ (7 ml). The organic layer was washed with a 10% solution of K₂CO₃, dried over Na₂SO₄ and concentrated to dryness to give the title compound as a white solid (190 mg, yield quantitative). ¹H NMR (400 MHz, CDCl₃) δ 2.27-2.34 (3H, m), 2.37-2.72 (8H, m), 4.79-4.82 (1H, m), 7.00 (2H, d), 7.21-7.26 (1H, m), 7.33-7.43 (1H, m), 7.44-7.67 (4H, m), 7.81-7.94 (2H, m), 8.37 (1H, d); Chiral HPLC [Chiralcel OD, 25×4.6 cm. Flow rate: 10.8 ml/min. UV detection: 225 nm. Mobil phase: nHexane/ethanol 70/30]: Rₜ=5.642 min (98.28% ee). Absolute stereochemistry determined using comparative vibrational circular dichroism (VCD), employing a closely related analog of known absolute stereochemistry (determined via ab-initio VCD analysis) as the comparator.

### Compound 3: (2R)-N-[(3,5-dichlorophenyl)methyl]-2-(4-methyl-1-piperazinyl)-2-(1-naphthalenyl)ethanamide hydrochloride

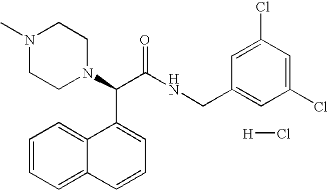

20 mg of Compound 2 ((2R)-N-[(3,5-dichlorophenyl)methyl]-2-(4-methyl-1-piperazinyl)-2-(1-naphthalenyl)ethanamide, 0.045 mmole) were dissolved in 450 μl of dry DCM in a dry flask under nitrogen. Then 45 μl of HCl 1M solution in Et₂O (0.045 mmole, Aldrich) were added. The solution was then stirred for 30 minutes. The solvents were removed under reduced pressure and the resulting crude solid was triturated with Et₂O. The solid was filtered and dried to give the title Compound as a sand colored solid (21.3 mg, quantitative). ¹H-NMR (CDCl₃, 400 MHz): δ 2.60-3.04 (4H, m), 2.76-283 (3H, m), 3.06-3.41 (4H, m), 4.18-4.30 (1H, m), 4.30-4.43 (1H, m), 4.83 (1H, br s), 6.80 (3H, d), 7.11-7.17 (1H, m), 7.44-7.61 (3H, m), 7.65-7.75 (1H, m), 7.84-7.94 (2H, m), 8.50 (1H, d), 12.53 (1H, br s); m/z (ES+): 442.3 [M+H]+.

### Biological Assays

### Cloning of the Ghrelin Receptor GHS-R

Human GHS-R was cloned from human hypothalamus cDNA and TOPO Ta cloned into pCR2.1. The sequence was confirmed. The full-length gene was transferred into pCDN for expression analysis. The sequence was confirmed again and the plasmid was electroporated into CHO cells. The clones were screened by FLIPR.

### Generation of Cells Transiently Expressing the Ghrelin Receptor GHS-R

HEK293T cells (HEK293 cells stably expressing the SV40 large T-antigen) were maintained in DMEM containing 10% (v/v) newborn calf serum and 2 mM glutamine. Cells were seeded in 60 mm culture dishes and grown to 60-80% confluency (18-24 h) prior to transfection with pcDNA3 containing the relevant DNA species using Lipofectamine reagent. For transfection, 3 μg of DNA was mixed with 10 μl of Lipofectamine in 0.2 mL of Opti-MEM (Life Technologies Inc.) and was incubated at room temperature for 30 min prior to the addition of 1.6 mL of Opti-MEM. For cotransfection experiments, 1.5 μg of each cDNA species was used. Cells were exposed to the Lipofectamine/DNA mixture for 5 h and 2 mL of 10% (v/v) newborn calf serum in DMEM was then added. Cells were harvested 48 h after transfection.

Membrane Preparation from Cultured Cells

All steps of the protocol are carried out at 4° C. and with pre-cooled reagents. The cell pellet was resuspended in 10 volumes of buffer A2 containing 50 mM N-2-hydroxyethylpiperazine-N′-2-ethanesulfonic acid (HEPES) (pH 7.40) supplemented with 10e-4M leupeptin (acetyl-leucyl-leucyl-arginal; Sigma L2884), 25 μg/mL bacitracin (Sigma B0125), 1 mM ethylenediamine tetra-acetic acid (EDTA), 1 mM phenylmethylsulfonyl fluoride (PMSF) and 2×10e-6M pepstain A (Sigma). The cells were then homogenised by 2×15 sec bursts in a 1 litre glass Waring blender, followed by centrifugation at 500 g for 20 mins. The supernatant was then spun at 48,000 g for 30 mins. The pellet was resuspended in 4 volumes of buffer A2 by vortexing for 5 secs, followed by homogenisation in a Dounce homogeniser (10-15 strokes). At this point the preparation was aliquoted into polypropylene tubes and stored at −70° C.

Compounds of the invention were tested for in vitro biological activity in accordance with the following GTPγS assays:

### GHS-R GTPγS Functional Agonist Assay Protocol

For each compound being assayed, in an Opti clear bottom 96 well plate, is added:—
(a) 20 μl of test compound (or 10 μl of guanosine 5′-triphosphate (GTP) (Sigma) as non-specific binding control) diluted to required concentration in assay buffer (20 mM N-2-Hydroxyethylpiperazine-N′-2-ethanesulfonic acid (HEPES)+100 mM NaCl+10 mM MgCl₂, pH adjusted to 7.4 with NaOH);
(b) 60 μl bead/membrane/GDP mix prepared by suspending wheat germ agglutinin-polyvinyltoluene (WGA-PVT) scintillation proximity assay (SPA) beads at 100 mg/mL in assay buffer followed by mixing with membrane (prepared in accordance with the methodology described above) and diluting in assay buffer to give a final volume of 60 μl which contains 10 μg protein and 0.5 mg bead per well-mixture was pre-mixed at 4° C. for 30 mins on a roller and just prior to addition to the plate, 10 μM final concentration of guanosine 5′ diphosphate (GDP) (Sigma; diluted in assay buffer) was added;
(c) 10 μl guanosine 5′ [γ35-S] thiotriphosphate, triethylamine salt (Amersham; radioactivity concentration=37 kBq/μl or 1 mCi/ml; Specific Activity 1160 Ci/mmol) diluted to 3.8 nM in assay buffer to give 0.38 nM final.

The plate was then incubated on a shaker at room temperature for 30 mins followed by centrifugation for 5 mins at 1500 rpm. The plate was read between 3 and 6 hours after completion of centrifuge run in a Wallac Microbeta counter on a 1 min normalised tritium count protocol. Data was analysed using a 4-parameter logistic equation. Basal activity used as minimum.

### GHS-R GTPγS Functional Antagonist Assay Protocol

Protocol as described above for functional agonist assay, except prior to addition of the [35-S], 10 ul of human Ghrelin solution in assay buffer was added to give a final concentration equating to EC80 (determined for each assay).

Compound 1 (as the trifluoroacetate salt) and Compound 3 were tested in the GHS-R GTPγS functional antagonist assay and found to give a pKi of greater than 8.0.

### Cloning of the Ghrelin Receptor GHS-R

Human GHS-R was cloned from human hypothalamus cDNA and TOPO Ta cloned into pCR2.1. The sequence was confirmed. The full-length gene was transferred into pCDN for expression analysis. The sequence was confirmed again and the plasmid was electroporated into CHO cells. The clones were screened by FLIPR.

### Generation of the GHS-R Bacmam Virus and Viral Titre Determination Virus Generation

The open reading frame of GHS-R was transferred from pCDN into pFastBacmam vector. This vector was used to generate recombinant baculoviruses in which the insect cell-specific polyhedrin promoter has been replaced with a mammalian cell-active promoter, in this case CMV. This was then used with the Bac to Bac expression system (Invitrogen). Briefly the vector was transformed into DH10 bac E. coli and the bacmid isolated from the transformed cells. The bacmid was then transfected into Sf9 insect cells grown in ExCell 420 (JRH) medium in 6-well dishes for the production of recombinant baculovirus particules.

The supernatant from these cells was harvested containing the recombinant GHS-R bacmam virus. This PO viral stock was then used to infect 200 mls of 1×10⁻⁶ cells/ml Sf9 cells in ExCell 420 medium to further amplify the virus and provide a P1 stock.

This P1 viral stock was then used to amplify a P2 viral stock of 10×1 litre erlemeyer shake flasks again harvesting the supernatant from the cells. This was then used to transduce mammalian cells for assay

### Viral Titre Determination

Viral titres were determined at all stages of the virus scale up with a plaque elisa method using a gp64 envelope protein monoclonal antibody.

SF9 cells were plated out into a 96 well plate and a dilution range of virus was added to the cells for 1 hour. The virus was removed and a 1% methylcellulose and media mix was added to the cells and incubated for 48 hrs. The cells were then fixed in a formaldehyde and acetone mix for 8 minutes. The cells were then washed with a phosphate buffered saline solution (PBS) and normal goat serum added for 25 mins. This was then removed and a gp64 monoclonal antibody added for 25 mins. The wells were then washed with PBS and a goat anti-mouse/HRP conjugated antibody added for 25 mins. The wells were again washed with PBS and True Blue peroxidase substrate solution (Kirkegaard & Perry Laboratories) added and incubated for 60 mins.

Individual wells were counted for blue foci and taking into account the dilution factor, the plaque forming units/ml of the virus was determined.

### Generation of U2OS Cells Transiently Expressing the Ghrelin Receptor GHS-R

24 hours prior to assay U2OS cells at confluence 100% were harvested and spun down. The supernatant was removed and the cells resuspended in media (DMEM+10% FBS+1% L-Glutamine). A cell count was performed using the Cedex instrumentation, and the concentration of cells was adjusted using media to give 200K cells per ml (10K cells/50 ul).

Human GHSR BACMAM virus was added to the cell suspension at an appropriate % volume (calculated for individual batches of BACMAM virus as viral titres vary). The transduced cell suspension was dispensed into FLIPR 384-well clear bottom plates, 50 ul per well. Cell plates were incubated at 37° C. overnight.

### Compound Preparation

### Method A

Master compound plates were prepared in 100% DMSO. 3 mM was the top concentration (giving 10 μM final concentration) and they were serially diluted 1 in 4. 1 ul from the master plate was transferred to a daughter plate, to which was added 50 μl of compound dilution (Tyrodes {Elga water+145 mM NaCl+5 mM KCl+20 mM HEPES+10 mM glucose+1 mM MgCl₂+1.5 mM CaCl₂}. For the agonist assay, the compound buffer also contained 0.1% BSA. This plate was used for the assay. Ghrelin was always prepared in buffer containing 0.1% BSA.

### Method B

Master compound plates were prepared in 100% DMSO. 3 mM was the top concentration (giving 10 μM final concentration) and they were serially diluted 1 in 4. 1 ul from the master plate was transferred to a daughter plate, to which was added 50 μl of compound dilution (HBSS {Elga water+137 mM NaCl+5 mM KCl+0.41 mMa KH2PO4(anhyd)+20 mM HEPES+5 mM glucose+0.81 mM MgSO4(anhyd)+1.3 mM CaCl₂+4.16 mM NaHCO3}. For the agonist assay, the compound buffer also contained 0.1% BSA. This plate was used for the assay. Ghrelin was always prepared in buffer containing 0.1% BSA.

GHSR Agonist BACMAM FLIPR Assay protocol

### Method X

Media was aspirated from cell plates, prepared according to method A above, using a cell washer (leaving 10 ul of media). Cells were immediately loaded with loading buffer (Tyrodes buffer—(Elga water+145 mM NaCl+5 mM KCl+20 mM HEPES+10 mM glucose+1 mM MgCl₂)+1.5 mM CaCl₂+0.714 mg/ml Probenecid (predissolved in 1 M NaOH)+0.25 mM brilliant black+2 uM Fluo 4 dye, and incubated at 37.5° C., 5% CO for 90 mins. The plates were placed in a FLuorimetric Imaging Plate Reader (FLIPR, Molecular Devices) where 10 μl of compound was added to the cells and fluorescence measurements were taken. Maximum changes in fluorescence were plotted as a percentage of the maximum response elicited by 300 nM hGhrelin and curves fitted using a 4-parameter logistic equation to generate pEC50 values. Intrinsic activity of the compounds was calculated by using the maximum asymptote of it's concentration response curve relative to the maximum asymptote of the hGhrelin concentration response curve.

### Method Y

Media was aspirated from cell plates, prepared according to method B above, using a cell washer (leaving 10 ul of media). Cells were immediately loaded with loading buffer (HBSS (Elga water+137 mM NaCl+5 mM KCl+0.41 Mm KH2PO4(anhyd)+20 mM HEPES+5 mM glucose+0.81 mM MgSO4(anhyd))+1.3 mM CaCl₂+4.16 mM NaHCO3+0.714 mg/ml Probenecid (predissolved in 1 M NaOH)+0.25 mM brilliant black+2 uM Fluo 4 dye, and incubated at 37.5° C., 5% CO for 90 mins. The plates were placed in a FLuorimetric Imaging Plate Reader (FLIPR, Molecular Devices) where 10 μl of compound was added to the cells and fluorescence measurements were taken. Maximum changes in fluorescence were plotted as a percentage of the maximum response elicited by 300 nM hGhrelin and curves fitted using a 4-parameter logistic equation to generate pEC50 values. Intrinsic activity of the compounds was calculated by using the maximum asymptote of it's concentration response curve relative to the maximum asymptote of the hGhrelin concentration response curve.

### GHSR Antagonist BACMAM FLIPR Assay Protocol

10 μl of compound was added to cell plates, prepared according to either Method A or B above, using an FX robot and the plate was then incubated (37.5° C., 5% CO2) for a further 30 min before being assayed on a FLIPR, where 10 μl of an EC80 concentration of hGhrelin was added to the cells and fluorescence measurements were taken. Maximum changes in fluorescence were plotted and curves fitted using a 4-parameter logistic equation to generate pIC50 values.

Compound 3 was tested in the GHSR Antagonist BACMAM FLIPR Assay (Method B) and found to give a pIC50 value of 8.0 (This data is thought to be accurate to within ±0.5 of the value stated).

Compound 3 was tested in the GHSR Agonist BACMAM FLIPR Assay (Method Y) and found to give a pEC50 value of <5.7 (This data is thought to be accurate to within ±0.5 of the value stated).

1. A compound of formula (I) or a pharmaceutically acceptable salt or solvate thereof:

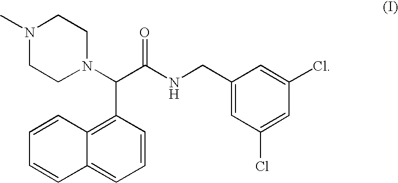

2. A compound according to claim 1 which is: 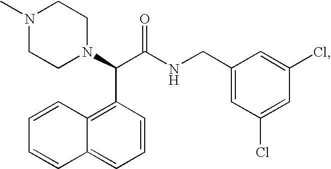
or a pharmaceutically acceptable salt or solvate thereof.

3. A process for the preparation of a compound of formula (I) or a pharmaceutically acceptable salt or solvate thereof as claimed in claim 1 which comprises:
(A) reacting a compound of formula (II) with an amine of formula (III) 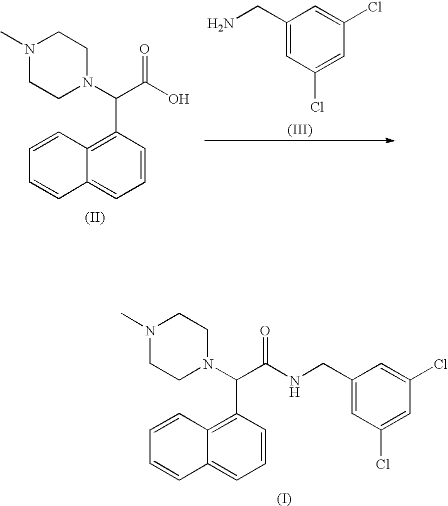
together in the presence of an amide coupling reagent and a suitable base in an aprotic solvent; followed by
(B) chiral separation if required.

4. A method of treatment of a mammal suffering from a disorder mediated by the ghrelin receptor, which comprises administering to said subject an effective amount of a compound of formula (I) or a pharmaceutically acceptable salt or solvate thereof according to claim 1.

5. A method of treatment of a mammal suffering from a binge eating disorder, which comprises administering to said subject an effective amount of a compound of formula (I) or a pharmaceutically acceptable salt or solvate thereof according to claim 1.

6-8. (canceled)

9. A pharmaceutical composition which comprises a compound of formula (I) or a pharmaceutically acceptable salt or solvate thereof according to claim 1 in admixture with one or more pharmaceutically acceptable carriers, diluents or excipients.

10. A pharmaceutical composition which comprises a compound of formula (I) or a pharmaceutically acceptable salt or solvate thereof according to claim 2 in admixture with one or more pharmaceutically acceptable carriers, diluents or excipients.

11. A method of treatment of a mammal suffering from a disorder mediated by the ghrelin receptor, which comprises administering to said subject an effective amount of a compound of formula (I) or a pharmaceutically acceptable salt or solvate thereof according to claim 2.

12. A method of treatment of a mammal suffering from a binge eating disorder, which comprises administering to said subject an effective amount of a compound of formula (I) or a pharmaceutically acceptable salt or solvate thereof according to claim 2.
